(19) United States
(12) Patent Application Publication
(10) Pub. No.: US 2012/0100555 A1
(43) Pub. Date: Apr. 26, 2012

(54) Synthesis And Use Of Fluorophore-Tagged Antimalarials

(76) Inventors: Martin James Lear, Singapore (SG); Kevin Shyong Wei Tan, Singapore (SG)

(21) Appl. No.: 13/381,616

(22) PCT Filed: Jun. 28, 2010
(86) PCT No.: PCT/SG10/00240

§ 371 Date: Dec. 29, 2011

Related U.S. Application Data
(60) Provisional application No. 61/221,304, filed on Jun. 29, 2009.

(51) Int. Cl. C12Q 1/68; G01N 33/559; C07D 405/12; G01N 33/567; C12Q 1/18

(52) U.S. Cl. 435/6.15; 435/7.2; 435/32; 546/163; 204/461

(57) ABSTRACT

This invention includes a fluorophore-tagged antimalarial represented by the following structural formula (1) or a salt thereof. This invention relates to the synthesis of fluorophore-tagged antimalarials and describes the synthesis of a fluorophore-tagged antimalarial. These fluorophore-tagged antimalarials can be used to image live cells to determine the location of the antimalarial in the cell, identify drug resistance and growth related pathways in Plasmodium isolates, identify new drug targets and chemo-sensitizers to reverse drug resistance.

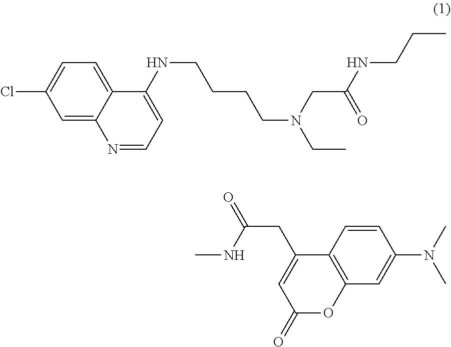

### RELATED APPLICATION

This application claims the benefit of U.S. Provisional Application No. 61/221,304, filed on Jun. 29, 2009. The entire teachings of the above application are incorporated herein by reference.

### BACKGROUND OF THE INVENTION

Malaria is a vector-borne infectious disease caused by protozoan parasites and is widespread in tropical and subtropical regions, including parts of the Americas, Asia and Africa. Of the five Plasmodium parasite species that can infect humans (P. falciparum, P. vivax, P. ovale, P. malariae and P. knowlesi), the most serious forms of the disease are caused by P. falciparum and P. vivax. Of the approximately 515 million people infected yearly, between one and three million people, the majority of whom are young children in Sub-Saharan Africa, die from the disease.

Before the emergence of drug-resistant Plasmodium strains in the 1960s, chloroquine (CQ) was a life-saving tool in the control of malaria (Greenwood B M, et al., Malaria. Lancet. 2005 Apr. 23-29; 365(9469):1487-98; Lalloo D G, et al., Lancet Infect Dis. 2006; 6(12):780-93). Today, only artemisinin-based drug combinations can effectively combat all parasitic forms of the disease (White, N. J. Malar J, 2008, 7 Suppl 1, S8). The biomolecular mechanisms of drug-resistance between differing strains of Plasmodium parasites and differing types of antimalarial agents are still poorly understood (White, N. J. Drug Resist Updat, 1998, 1, 3-9; Bloland P. B., et al., Bull World Health Organ., 2000, 78, 1378-1388).

The current armament of approved anti-malarial drugs is limited to only a few targets within the human malaria parasite. Growing widespread resistance to current drugs is prompting the development of new antimalarial agents that have new biological targets. While identification of new drug targets could facilitate the development of effective antimalarials, new targets can be difficult to identify. Determination of cellular accumulation of the antimalarials such as chloroquine could help understanding drug action and may lead to identification of new targets. Furthermore, a means to rapidly identify drug resistance in the field could lead to quicker diagnosis and the administration of appropriate chemotherapy. Therefore, a need exists to develop methods to examine the interaction of antimalarials within the Plasmodium parasite in order to elucidate new pathways that can be exploited for the successful treatment of malaria.

### SUMMARY OF THE INVENTION

This invention relates to the synthesis of fluorophore-tagged antimalarials and describes reagents, conditions, methods, routes, and strategies to make fluorescent-tagged antimalarials. A fluorescent-tagged antimalarial has been synthesized and used to image the accumulation of the antimalarial within a live cell. Fluorophore-tagged antimalarials can be used to image live cells to locate the antimalarial within or on the cell, screen for drug resistance and identify growth-related pathways in Plasmodium isolates, and identify new drug combinations and new drug targets. The invention has many advantages: the fluorescent tag is not radioactive, the tag is small, and non-toxic, and therefore should not interfere with cellular processes.

One embodiment of the invention is a compound for use in malarial research comprising a coumarin-tagged antimalarial, wherein the coumarin is optionally substituted and is connected to the antimalarial through a linker.

Another embodiment of the invention is a compound comprising a coumarin-tagged antimalarial comprising coumarin connected to the antimalarial through a linker, wherein the coumarin and linker are represented by the following structural formula:

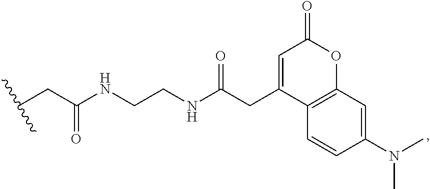

or salts thereof.

Another embodiment of the invention is a compound represented by the following structural formula:

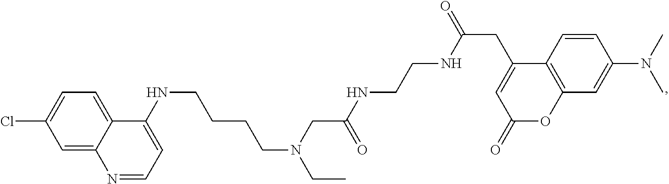

or a salt thereof.

Another embodiment of the invention is a compound represented by the following structural formula:

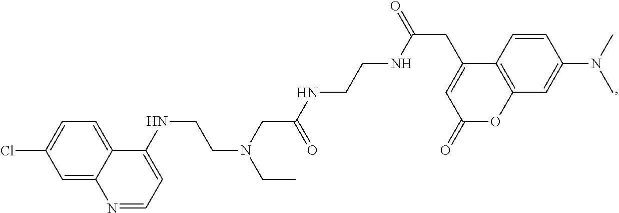

or a salt thereof.

Another embodiment of the invention is a compound represented by the following structural formula:

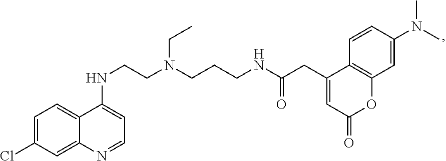

or a salt thereof.

Another embodiment of the invention is a compound represented by the following structural formula:

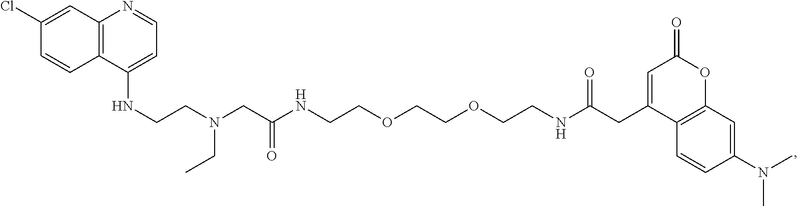

or a salt thereof.

Another embodiment of the invention is a method for the identification of a target in a cell, comprising the steps of exposing the cell to a fluorophore-tagged antimalarial described herein, allowing the fluorophore-tagged antimalarial to bind to its target; capturing the fluorophore-tagged antimalarial bound to the target on an immobilized affinity ligand specific for the fluorophore tag; and identifying the target bound to the fluorophore-tagged antimalarial.

Another embodiment of the invention is a method for identification of a target in a cell, comprising the steps of exposing the cell to the fluorophore-tagged antimalarial of the following structural formula:

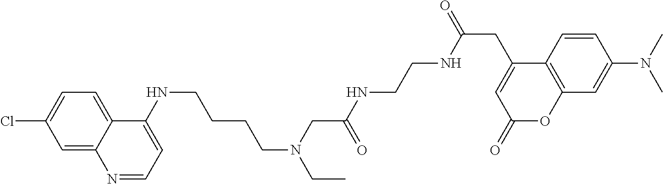

or a salt thereof; allowing the fluorophore-tagged antimalarial to bind to its target, capturing the fluorophore-tagged antimalarial bound to the target on an immobilized affinity ligand specific for the fluorophore tag; and identifying the target bound to the fluorophore-tagged antimalarial.

Another embodiment of the invention is a method for determining the location of a fluorophore-tagged antimalarial within or on a cell comprising the steps of: exposing the cell to a fluorophore-tagged antimalarial described herein; and assaying the cell to determine the location of the fluorophore-tagged antimalarial within or on the cell.

Another embodiment of the invention is a method for determining the location of a fluorophore-tagged antimalarial within or on a cell comprising the steps of: exposing the cell to the fluorophore-tagged antimalarial of the following structural formula:

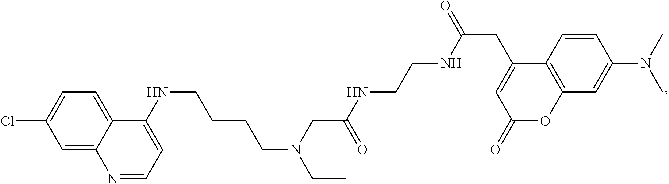

or a salt thereof; and assaying the cell to determine the location of the fluorophore-tagged antimalarial within or on the cell.

One more embodiment of the invention includes a method of preparing a fluorophore-tagged antimalarial represented by the following structural formula:

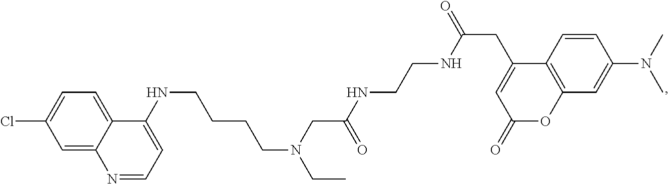

or a salt thereof; said method comprising the step of reacting a compound represented by the following structural formula:

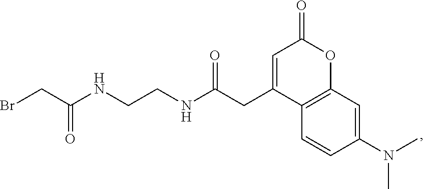

or a salt thereof, with an antimalarial represented by the following structural formula:

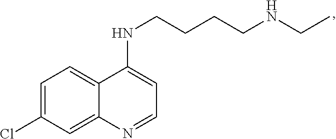

or a salt thereof, thereby forming the fluorophore-tagged antimalarial.

### BRIEF DESCRIPTION OF THE DRAWINGS

FIG. 1 is a graph showing the concentration-dependent accumulation of coumarin-labeled chloroquine (CM-CQ) in different cellular compartments in parasite cells.

FIG. 2 is a graph showing the IC₅₀ for commercial chloroquine (CQ), coumarin (CM) and coumarin-labeled chloroquine (CM-CQ). Parasitized cultures were incubated for 48 hours with various concentrations of the test compounds and, thereafter, parasitemia was determined using Hoechst staining and flow cytometric analyses.

FIG. 3 is a graph showing the dose-dependent study of chloroquine's effect on JC-1 loss (black line) and CaspaTag staining (grey line). Parasitized cultures were incubated for 8 hours with different concentrations of chloroquine. (n=2)

FIG. 4 is a synthetic scheme for CM-CQ (Compound 2).

FIG. 5 is a synthetic scheme for Compound 8.

FIG. 6 is a synthetic scheme for Compound 13.

FIG. 7 is a synthetic scheme for Compound 16.

FIG. 8 is a graph showing the effects of chemo-sensitizers on coumarin-conjugated chloroquine (CM-CQ) accumulation in Plasmodium falciparum parasites. Data were obtained by flow cytometry.

FIG. 9 is a graph showing the effects of chemo-sensitizers on coumarin-conjugated chloroquine (CM-CQ) accumulation in Plasmodium falciparum parasites, obtained by spectrofluorometer plate readings.

### DETAILED DESCRIPTION OF THE INVENTION

Malaria is an infection of red blood cells with the single-celled parasite Plasmodium. Malaria parasites are transmitted by female Anopheles mosquitoes. The parasites multiply within red blood cells, causing symptoms that include symptoms of anemia (light headedness, shortness of breath, tachycardia), as well as other general symptoms such as an enlarged spleen, fatigue, fever, chills, nausea, flu-like illness, and in severe cases, coma and death.

The life cycle of malaria parasites in the human body starts when a mosquito infects a person by taking a blood meal. Next, sporozoites enter the bloodstream, and migrate to the liver. They infect liver cells (hepatocytes), where they multiply into merozoites, rupture the liver cells, and escape back into the bloodstream. Then, the merozoites infect red blood cells, where they develop into ring forms, then trophozoites (a feeding stage), then schizonts (a reproduction stage), then back into merozoites. Sexual called gametocytes are also produced, which, if taken up by a mosquito, will infect the insect and continue the life cycle.

One embodiment of the invention is a fluorophore-tagged antimalarial. A “fluorophore-tagged antimalarial” is a compound that includes both a fluorophore and an antimalarial moiety. Use of fluorophore-tagged antimalarials are anticipated to not only elucidate promising targets for therapeutic drugs, but also enable a greater understanding of the physiological role of growth-related pathways in unicellular organisms. In particular, malaria research includes cellular localization studies of antimalarials, and elucidation of drug targets and growth-related pathways. Fluorophore-tagged antimalarials can also be used to understand mechanisms of drug resistance in Plasmodium species. In particular, the localization of the labeled drug may be predicted to be different between sensitive and resistant strains. By extension, this difference in fluorescence between sensitive (high fluorescence) and resistant (low fluorescence) strains allows the use of the labeled drug to identify other drugs that reverses resistance by screening for such drugs, termed chemoreversal agents or chemosensitizers, in the presence of the fluorescent-tagged antimalarial, that result in high fluorescence in resistant parasites.

As used herein, “fluorophore” is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. A fluorophore can be selected because it can be used during the 48 hours erythrocytic cycle, is photostable, can be continuously imaged in live cells, and has high fatigue strength. High fatigue strength refers to the longevity of fluorescence over long time periods (>48 hours) and non-destruction of fluorophore unit either biologically or photochemically).

Certain fluorophore tags, such as coumarin, are biologically inert and therefore non-toxic to malaria parasites. Thus, cellular responses to the fluorophore-tagged antimalarial should be attributable to the antimalarial component.

In a specific embodiment, the fluorophore is coumarin. Coumarin is represented by the following structural formula:

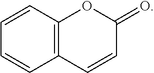

Derivatives of coumarin are contemplated. For example, any substitutable hydrogen on a ring carbon atom may be substituted by halo, OH, NO₂, CN, COOH, COO(C₁-C₆alkyl), amino, (C₁-C₆)alkyl, (C₁-C₆)alkoxy, (C₁-C₆)alkylamino, or di(C₁-C₆)alkylamino, wherein the (C₁-C₆)alkyl and (C₁-C₆)alkoxy in any of the preceeding groups may be optionally substituted with halogen, (C₁-C₆)alkoxy, OH, NO₂, COOH, COO(C₁-C₆alkyl), or CN. In a specific embodiment, coumarin is substituted with dimethylamino.

In another embodiment, the substituent can be used to form the linker and thus be modified to connect the fluorophore such as coumarin to the antimalarial during the synthesis of the fluorophore-tagged antimalarial. For example, the substituents that can be modified during the synthesis to form the linker include a carboxylic acid (e.g. —COOH, —CH₂COOH, —(CH₂)₂COOH, —(CH₂)₃COOH, —(CH₂)₄COOH, etc., wherein any of the substitutable hydrogen on a carbon atom may be substituted as above) or an ester. In one embodiment, the fluorophore is coumarin substituted with dimethylamino and —CH₂COOH.

In one embodiment of the invention, the coumarin derivative to be incorporated into the fluorophore-tagged antimalarial is 7-dimethylaminocoumarin-4-acetic acid, which is represented by the following structural formula:

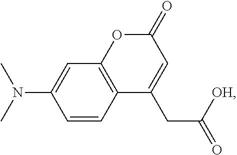

or a salt thereof.
7-dimethylaminocoumarin-4-acetic acid is known in the art. See Goddard J. P. and J. L. Reymond, Trends. Biotechnol. 2004, 22, 363; Lim N. C. and C. Bruckner, Chem. Commun. 2004, 1094; Trenor S. R. et al., Chem. Rev. 2004, 104, 3059; Lim N. C. et al., Inorg. Chem. 2005, 44, 2018; Gonsalves, M. S. T. Chem. Rev. 2009, 109, 190.
7-dimethylaminocoumarin-4-acetic acid has many suitable properties including:
photophysical activity (Jarzeba, W. G. C. et al., J. Phys. Chem. 1998, 92, 7039);
non-cytotoxicity (Alexander M. D., et al., Chembiochem. 2006, 7, 409);
small-size (Fritz M. G., D. Seebach, Helv. Chim. Acta 1998, 81, 2414); and
water solubility (Souto A. A., et al., Angew. Chem. Int. Ed. Engl. 1995, 34, 2710).

Fluorophores other than coumarin can be used in this invention. Examples of fluorophores that can be incorporated in a fluorophore-tagged antimalarial include:

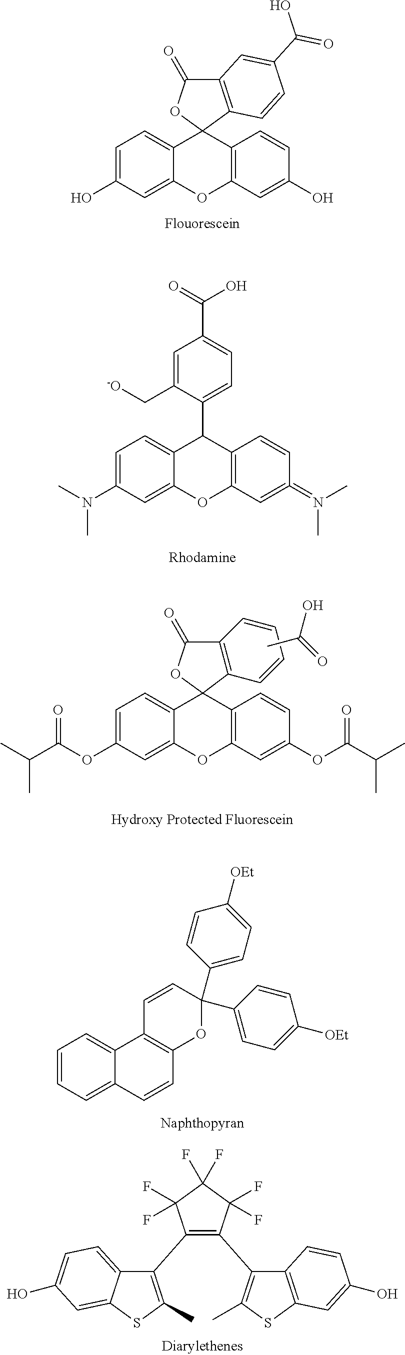

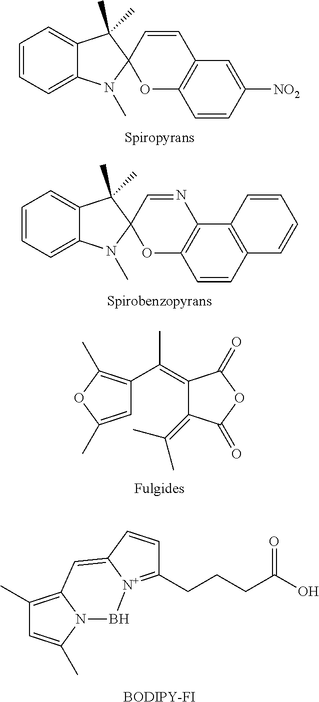

The fluorophores above can be modified to facilitate coupling of the fluorophore to the linker and/or the antimalarial.

A fluorophore-tagged antimalarial including coumarin as the fluorophore is referred to as a “coumarin-tagged antimalarial.” One embodiment of the invention is a compound comprising a coumarin-tagged antimalarial, wherein the coumarin is optionally substituted, as described above, and is connected to the antimalarial through a linker.

In the fluorophore-tagged antimalarial, the fluorophore is attached to the antimalarial (as described below) through a linker to form the fluorophore-tagged antimalarial.

The linker is a covalent bond, or an alkylene group, i.e., —(CH₂)ₓ— where x is a positive integer (for example, from 1 to about 18, more specifically between 1 and about 10, or more specifically between 2 and 8, or more specifically between 4 and 8.) Any substitutable hydrogen on a carbon atom in the linker may be substituted by halo, OH, NO₂, CN, COOH, COO(C₁-C₆alkyl), amino, (C₁-C₆)alkyl, (C₁-C₆)alkoxy, (C₁-C₆)alkylamino, or di(C₁-C₆)alkylamino, wherein the (C₁-C₆)alkyl and (C₁-C₆)alkoxy in any of the preceeding groups may be optionally substituted with halogen, (C₁-C₆)alkoxy, COOH, COO(C₁-C₆alkyl), OH, NO₂ or CN. In addition, the carbon chain of the alkylene linker can be optionally interrupted with one or more groups including an alkene, alkyne, phenylene, ether, thioether, amine, (C₁-C₆)alkylamino, di(C₁-C₆)alkylamino, ester, thioester, amide or carbamate. In a specific example, the linker is an alkylene group that is interrupted with one or more amide groups. In a particular example, the alkylene group is interrupted with two amide groups.

In a particular embodiment, the linker is represented by the following structural formula:

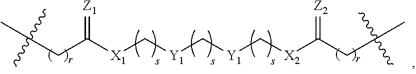

wherein each r, s, and t are independently and optionally 0, 1, 2, or 3, w is 1, and X₁, X₂, Y₁ and Y₂ are independently CR₂, O, S, or NR, wherein R is an alkyl group, an acyl group, an aryl group, a cycloalkyl group, or a heterocycle group. Alternatively, R can form an aryl group, a cycloalkyl group or a heterocycle group with another carbon atom within the linker. Z₁ and Z₂ is independently O, S or H₂.

In a specific embodiment, the linker is represented by the following structural formula:

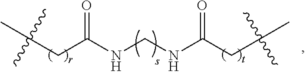

wherein r, s, and t are independently and optionally 0, 1, 2, or 3.
The symbol

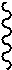

indicates the point of connection to fluorophore and the antimalarial. Either the fluorophore or the antimalarial can be attached to either the left side or the right side of the linker. Any substitutable hydrogen on a carbon atom of this linker may be substituted by halo, OH, NO₂, CN, COOH, COO(C₁-C₆alkyl), amino, (Cᵣ C₆)alkyl, (C₁-C₆)alkoxy, (C₁-C₆)alkylamino, or di(C₁-C₆)alkylamino, wherein the (C₁-C₆)alkyl and (C₁-C₆)alkoxy in any of the preceeding groups may be optionally substituted with halogen, (C₁-C₆)alkoxy, COOH, COO(C₁-C₆alkyl), OH, NO₂ or CN. In a specific embodiment, coumarin is substituted with dimethylamino. Any substitutable hydrogen on a nitrogen atom may be substituted by 1 to 3 groups selected from (C₁-C₆)alkyl, optionally substituted with halogen, (C₁-C₆)alkoxy, COOH, COO(C₁-C₆alkyl), OH, NO₂ or CN. In a specific embodiment, r and t are independently 1 or 2 and s is 2 or 3. In a particular embodiment, the linker is

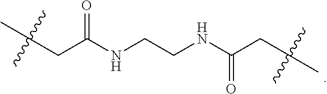

In a particular embodiment, the linker is

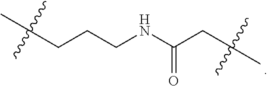

In a particular embodiment, the linker is

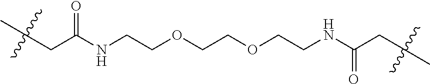

In a specific embodiment, the coumarin-tagged antimalarial includes coumarin and linker, which are represented by the following structural formula:

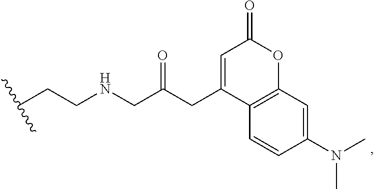

or salts thereof.

As used herein, “antimalarial” or “antimalarial compound” means a compound used to treat or prevent malaria in a patient. An antimalarial compound may be used therapeutically, may be in development for therapeutic use, may be known to have antimalarial properties, but not used therapeutically, or may have an unknown effect in addition to killing the parasite. Non-therapeutic uses of antimalarials include malarial research. Examples of antimalarials include quinine, atovaquone, chloroquine, cycloguanil, hydroxycholoroquine, amodiaquine, pyrimethamine, sulphadoxine, proguanil, mefloquine, halofantrine, pamaquine, primaquine, artemesinin, artemether, artesunate, artenimol, lumefantrine, dihydroartemisinin, piperaquine, arteether, doxycycline and clindamycin.

Antimalarial derivatives are also included in the invention. Antimalarial derivatives are antimalarial compounds that have been modified as compared to the antimalarial. For example, an antimalarial can be derivatized to incorporate a chemical moiety into the antimalarial such as a hydroxyl, halogen, a carboxylic acid, an ester, an alkoxy or a alkyl or any combination thereof. An antimalarial can be derivatized to remove a chemical moiety present in the antimalarial such as a hydroxyl, halogen, a carboxylic acid, an ester, an alkoxy or an alkyl or any combination thereof. An antimalarial can also be modified by, for example, by shortening or lengthening alkyl groups within the antimalarial to form an antimalarial derivative. Chemical moieties can be added, removed or modified independently of each other. Specifically, the antimalarial is chloroquine or a derivative thereof. Chloroquine is represented by Structural Formula I and a derivative of chloroquine is represented by Structural Formula II:

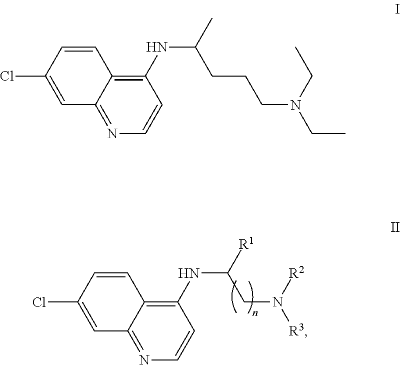

and salts thereof. In Structural Formula II, n is 0, 1, 2, 3, or 4; R¹, R² and R³ are independently selected from is H, (C₁-C₆)alkyl, halo(C₁-C₆)alkyl, (C₁-C₆)alkoxy or (C₁-C₆)cycloalkyl; and R⁴ is H, halogen, haloalkyl, hydroxy, CN or NO₂. R² and R³ may be used for attachment to the linker or fluorophore. In one embodiment of Structural Formula II, R¹ is H; R² is ethyl, R³ attaches to a linker; and R⁴ is Cl. In a specific embodiment of Structural Formula II R¹ is H; R² is ethyl, R³ attaches to a linker; R⁴ is Cl; and n is 2.

In addition to antimalarials, compounds without established antimalarial properties are also contemplated. These compounds may be those relevant for understanding of nutritional requirements and signaling events within the parasite.

In a specific embodiment, coumarin-tagged antimalarial is coumarin-tagged chloroquine, or a salt thereof, wherein the coumarin is optionally substituted.

In a particular embodiment of the invention, the coumarin-tagged chloroquine is represented by the following structural formula:

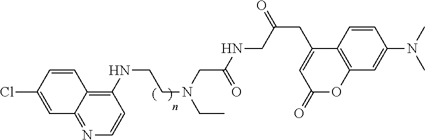

or a salt thereof, wherein n is independently and optionally 0, 1, 2, or 3.

In another particular embodiment of the invention, the coumarin-tagged chloroquine is represented by the following structural formula:

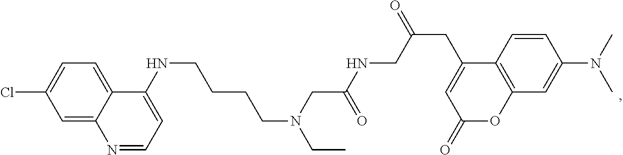

or a salt thereof.

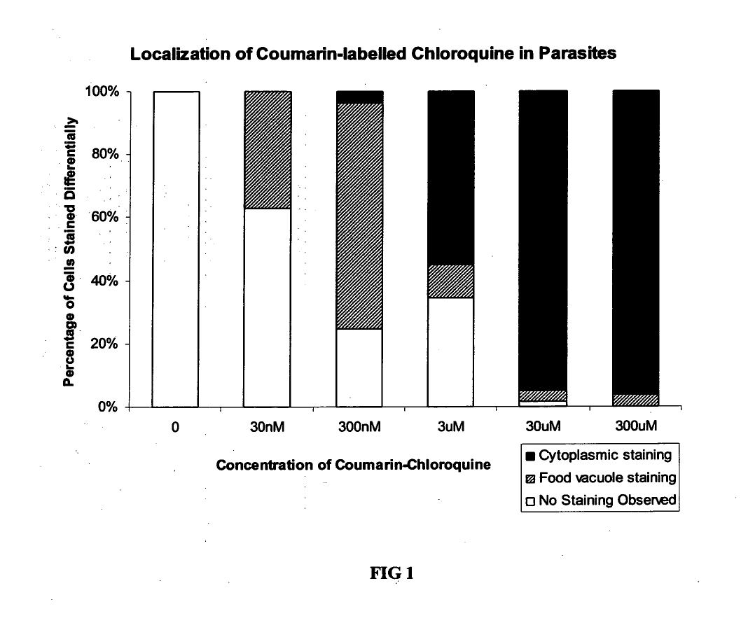

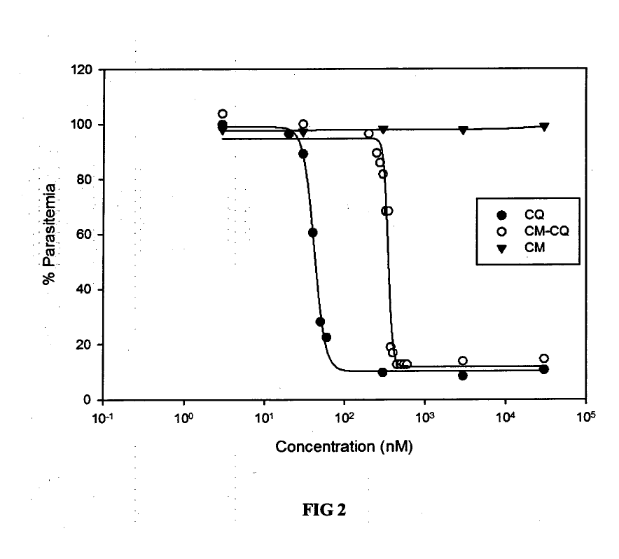

Fluorophore tags for use in the invention, such as coumarin, are biologically inert and therefore any cellular response should be attributable to antimalarial attached to the tag. As seen in FIG. 2, the coumarin-tagged chloroquine (CM-CQ) has activity comparable to chloroquine (CQ). The similarity of the activities CQ and CM-CQ allows for live imaging of cells to determine cellular localization (FIG. 1) of the drug as a means to understand drug action and resistance in Plasmodium species.

Another embodiment of the invention is a method for identification of a target in a cell, comprising the steps of exposing the cell to a fluorophore-tagged antimalarial described herein, allowing the fluorophore-tagged antimalarial bind to its target, and capturing the fluorophore-tagged antimalarial bound to the target with an affinity ligand (in immobilized or free form) specific for the fluorophore tag; and identifying the target bound to the fluorophore-tagged antimalarial. The affinity ligand can be an antibody specific for the fluorophore.

As used herein, a “target” is the biological molecule to which the antimalarial binds. A target includes metabolic by-products, enzymes, receptor and structural proteins, nucleic acids, signaling and membrane molecules.

An immobilized affinity ligand is used in a pull-down assay which also includes affinity chromatography. Affinity chromatography is a chromatographic method of separating biochemical mixtures, based on a highly specific biologic interaction such as that between antigen and antibody, enzyme and substrate, or receptor and ligand. Specifically, targets of the antimalarials can be identified or confirmed by the use of a fluorophore-tagged antimalarial in a pull-down assay using antibodies directed against fluorescent tags. The method comprises the steps of exposing the cell to the fluorophore-tagged antimalarial, allowing the fluorophore-tagged antimalarial to interact and bind to its target, capturing the fluorophore-tagged antimalarial, now bound to its target, on an immobilized affinity ligand specific for the fluorophore tag; and identifying the target bound to the fluorophore-tagged antimalarial.

In particular, immunoprecipitation (IP) protocols would begin by treating live infected cells (incorporating a Plasmodium species) with the coumarin-tagged antimalarial. The cells are washed with RIPA buffer (the medium used for IP protocols), lysed in the presence of protease inhibitors, and sub-cellular fractionated. This entire process would be monitored by fluorescence spectroscopy to minimize the loss in the IAF-labeled probe during this process. The isolated sub-cellular fractions are then subjected to affinity chromatography using anti-IAF mAb agarose resin. Calibrations of this method can be conducted using both engineered and native systems, indicating that the method can be used to isolate specific proteins at a concentration greater than 500 pg/ml from lysates containing a net protein content of 1 mg/ml.

The IP method provides advantages as compared to the biotin-strepavidin methods as one can effectively competitively-release the proteins readily from the resin by elution with 0.05 mM 7-dimethylaminocoumarin-4-acetic acid in phosphate buffer (PBS) or a gel loading buffer. This allows gels to return immunoprecipitated proteins without background from native interactions (as commonly seen in biotin/streptavidin procedures) or antibody contamination (arising by the conventional boiling of IP resins to release their protein content). Concentration of eluent to 20-50 μl by means of spin concentration with 5,000 MWCO removes all small molecules. After checking the protein content by spectrophotometrically with coumarin-labeled fluorescence, samples with greater than 10 ng/mL or protein are pooled and subjected to SDS page gel analysis and western blotting using the anti-coumarin mAb for detection.

The fluorophore, such as coumarin, may be an immunoaffinity-fluorescent (IAF) tag. Thus, in another embodiment of the invention, the coumarin-tagged antimalarial compound is used in co-immunopreciptation (co-IP) studies to identify biomolecules. Methods of performing co-immunoprecipitation experiments are known in the art. Representative examples of co-IP studies are described in Hughes C. C., et al., Angew. Chem. Int. Ed. Engl., 2009, 48, 728; Rodriguez A. D., et al., J. Am. Chem. Soc. 2008, 130, 7256; and Alexander M. D., et al., Chembiochem. 2006, 7, 409, the entire teachings of which are incorporated herein by reference.

As used herein “exposing the cell to the fluorophore-tagged antimalarial” means the cell and the fluorophore-tagged antimalarial are present in the same container or in the same solution and may come into contact. Exposing the cell the fluorophore-tagged antimalarial includes adding the fluorophore-tagged antimalarial, either in solution or as a solid, to the culture media used to cultivate the cells.

The fluorophore-tagged antimalarial may be added at any time during the cultivation. For example, the fluorophore-tagged antimalarial can be added to the media before the cells are added, can be added at the time the cells are added, or can be added after the cells are added. The exposure period can be the entire time of the cultivation of the cells or can be a shorter period than the cultivation of the cells. For example, the fluorophore-tagged antimalarial can be added to the media before or when the cells are added and left in the culture media until the cells are harvested. Alternatively, the fluorophore-tagged antimalarial can be added after the cells are added including after 5 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 16 hours, 20 hours, 24 hours, 32 hours, 40 hours, or 48 hours after. Alternatively, the fluorophore-tagged antimalarial can be added after the cells are synchronized (for example, the parasites are all at the ring stage). Synchronization techniques include, for example, sorbitol synchronization to select for ring-stage parasites. (See Lambros, C., and Vanderberg, J. P. (1979) J Parasitol 65, 418-420, the entire teachings of which are incorporated herein by reference.) The exposure can be ended by removing the media from the cells and adding new media that does not contain the fluorophore-tagged antimalarial. The exposure time can depend upon the length of time needed to achieve a biological result such as cell death.

As used herein “capturing” is a chemical or physical process by which the fluorophore-tagged antimalarial bound to the target is separated from the cell or cell debris following lysis of the cell. For example, the cells can be collected from the media through centrifugation. The cells can be lyzed by routine techniques, and thereby release the cell contents including the fluorophore-tagged antimalarial bound to the target into a solution.

The fluorophore-tagged antimalarial bound to the target in the solution (or “mobile phase”) is separated from other molecules in solution based on differences in chemical or physical interaction with a stationary material (solid phase). Affinity chromatography (also called affinity purification) makes use of specific binding interactions between the fluorophore of the fluorophore-tagged antimalarial and the stationary material. A particular ligand is chemically immobilized or “coupled” to a solid support so that when the solution containing a fluorophore-tagged antimalarial is passed over the column, only those molecules having specific binding affinity to the ligand (e.g., the fluorophore-tagged antimalarial bound to the target) are purified. Affinity purification generally involves the following steps: Incubate crude sample with the immobilized ligand support material to allow the target molecule in the sample to bind to the immobilized ligand. Wash away nonbound sample components from solid support. Elute (dissociate and recover) the target molecule from the immobilized ligand by altering the buffer conditions so that the binding interaction no longer occurs.

Another embodiment of the invention is a method for identification of a target in a cell, comprising the steps of exposing the cell to the fluorophore-tagged antimalarial of the following structural formula:

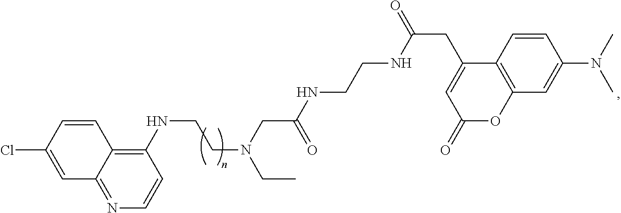

or a salt thereof, wherein n is independently and optionally 0, 1, 2, or 3; allowing the fluorophore-tagged antimalarial to bind to its target, capturing the fluorophore-tagged antimalarial bound to the target on an immobilized affinity ligand specific for the fluorophore tag; and identifying the target bound to the fluorophore-tagged antimalarial.

Another embodiment of the invention is a method for identification of a target in a cell, comprising the steps of exposing the cell to the fluorophore-tagged antimalarial of the following structural formula:

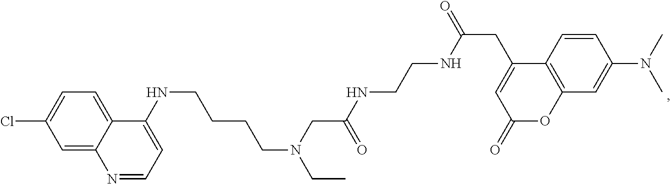

or a salt thereof; allowing the fluorophore-tagged antimalarial to bind to its target, capturing the fluorophore-tagged antimalarial bound to the target on an immobilized affinity ligand specific for the fluorophore tag; and identifying the target bound to the fluorophore-tagged antimalarial.

The target can be identified by routine methods known to those of skill in the art including mass spectrometry (for example, electrospray ionization (ESI) and matrix-assisted laser desorption/ionization (MALDI)), de novo protein sequencing, and gel electrophoresis. In one embodiment of the invention, the target is identified by N-terminal or internal protein sequencing, mass spectrometry, gel electrophoresis, or gene silencing with siRNA (See Natt F. siRNAs in drug discovery: target validation and beyond. Curr Opin Mol. Ther. 2007 June; 9(3):242-247, the entire teachings of which are incorporated herein by reference) or RNAi. (See Kourtidis A, et al. RNAi applications in target validation. Ernst Scheririg Res Found Workshop. 2007; (61):1-21, the entire teachings of which are incorporated herein by reference.)

For ESI, intact proteins are ionized and then introduced to a mass analyser. For MALDI, proteins are enzymatically digested into smaller peptides using a protease such as trypsin. Subsequently these peptides are introduced into the mass spectrometer and identified by peptide mass fingerprinting or tandem mass spectrometry (e.g. nano-LC/MS/MS analysis). Additional methods for target validation include gene silencing with siRNA or RNAi.

Another embodiment of the invention is a method for determining the location of a fluorophore-tagged antimalarial within or on a cell comprising the steps of exposing the cell to a fluorophore-tagged antimalarial described herein; and assaying the cell to determine the location of the fluorophore-tagged antimalarial within or on the cell.

Another embodiment of the invention is a method for determining the location of a fluorophore-tagged antimalarial within or on a cell comprising the steps of: exposing the cell to the fluorophore-tagged antimalarial of the following structural formula:

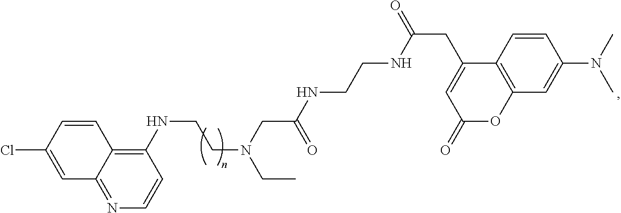

or a salt thereof, wherein n is independently and optionally 0, 1, 2, or 3 and assaying the cell to determine the location of the fluorophore-tagged antimalarial within or on the cell.

Another embodiment of the invention is a method for determining the location of a fluorophore-tagged antimalarial within or on a cell comprising the steps of: exposing the cell to the fluorophore-tagged antimalarial of the following structural formula:

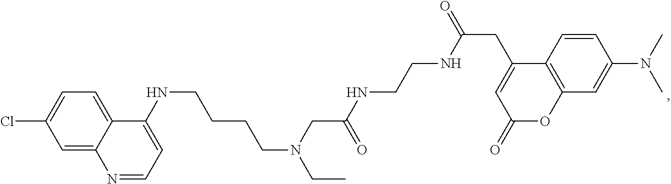

or a salt thereof, and assaying the cell to determine the location of the fluorophore-tagged antimalarial within or on the cell.

One embodiment of the invention involves use of the compounds of the invention as a screening tool to identify compounds that can alter chloroquine resistance. This is based on the principle that chloroquine sensitive malaria strains take up more fluorescent drug (as they would the natural unlabeled compound) while resistant strains are able to pump the drug out, resulting in less fluorescence. If a co-administered drug is able to reverse the pump effect, then an increase in fluorescence would be expected in the resistant strains. Based on this principle, the invention provides a method for the identification of compounds that can alter resistance to chloroquine in a resistant malaria parasite, comprising: exposing a chloroquine resistant cell to a test compound, adding the fluorophore-tagged antimalarial of the following structural formula:

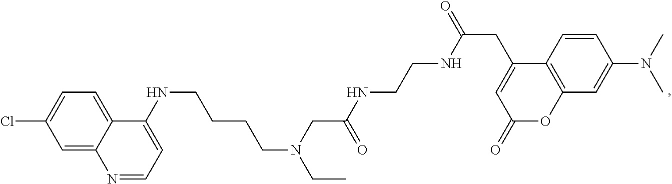

or a salt thereof to the parasite previously exposed to the test compound, and assaying the cell to determine a change in a fluorescence signal. An increase in the fluorescence signal from a baseline is indicative of a compound that can alter chloroquine resistance. If there is no change in the fluorescence signal from a baseline that is indicative of a compound that cannot alter chloroquine resistance.

Another embodiment of the invention involves use of the compounds of the invention as a screening tool to identify chloroquine resistant malaria strain. Based upon this embodiment, the invention provides a method of identification of a chloroquine resistaiit strain, comprising: exposing a strain to the fluorophore-tagged antimalarial of the following structural formula:

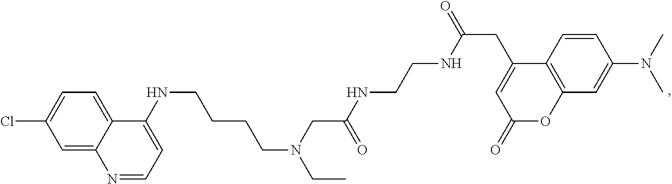

or a salt thereof; and assaying the strain to determine if the fluorophore-tagged antimalarial bound to the strain, by determining a change in a fluorescence signal, wherein a decrease in the fluorescence signal from baseline is indicative of the chloroquine resistant strain.

As used herein “assaying” refers to a determination of the quantity or location, or both of the fluorophore-tagged antimalarial. “Visualizing” is a method of assaying. As used herein “to alter resistance to chloroquine in a resistant parasite” means to decrease the parasite's resistance to chloroquine, by increasing in any amount, the uptake of chloroquine by the parasite, resulting in increase of the fluorescence signal from baseline. As used herein “baseline” means a standard, predetermined florescence signal, for example, thefluorescence signal of a CQ-susceptible strain exposed to CM-CQ can be used as a baseline.

Cellular localization of antimalarials can be identified by the use of a fluorophore-tagged antimalarial in bioimaging of live, fixed cells or cell lysates derived thereof from fixed or dead cells. The method comprises the steps of exposing the cell to the fluorophore-tagged antimalarial, allowing the fluorophore-tagged antimalarial to accumulate within or on the cell, and viewing the fluorescence emitted from the fluorophore tag. The fluorescence emitted can be assayed by techniques known to those of skill in the art and include fluorescence and confocal microscopy and flow cytometry.

In one embodiment, the cells are parasite infected red blood cells. The parasites include those of the Plasmodium species. In particular, four human Plasmodium parasite species: P. falciparum, P. vivax, P. ovale and P. malariae, the zoonotic primate species P. knowlesi and the three major rodent species P. yoelii, P. berghei and P. chabaudi are specific cell types. The parasites invade and reside in the red blood cells of the host, and therefore “cell” refers to the parasite cell within the host red blood cell. The cells of the host are herein referred to as “host cell,” or “host red blood cell”, or “red blood cell.” The methods described herein include parasite cells within the host cell.

The methods described herein may optionally include a further step of comparing the response of sets of cells exposed to different concentrations of the antimalarial or for different time periods. These methods may also be used when the parasite cultures are synchronized to be in one stage of its life stage (e.g., all cells are predominantly rings, etc.) or the parasite cultures are unsynchronized. A “set of cells” includes distinct cultures of the same parasite isolates (e.g., distinct cultures of P. falciparum 3D7); distinct cultures of different parasite isolates (e.g., distinct cultures of P. falciparum 3D7 and K1); or distinct cultures of the different parasite species (e.g., distinct cultures of P. falciparum and P. vivax); or any combination thereof.

The methods described herein may optionally include a further step of lysing red blood cells to liberate the parasite cell. In some embodiments, the parasite cell may also be lysed.

Drug resistance of Plasmodium isolates can be identified by the use of a fluorophore-tagged antimalarial. For example, antimalarials may distribute differently in Plasmodium isolates that are resistant to the antimalarial as compared to Plasmodium isolates that are sensitive to that antimalarial.

As used herein, “antimalarial-sensitive isolate” or “antimalarial-sensitive” means that the viability of the isolate is reduced or eliminated in the presence of the antimalarial. An isolate that is sensitive to an antimalarial is susceptible to the biological effects of that antimalarial. For example, a chloroquine-sensitive isolate is not viable in the presence of chloroquine. P. falciparum isolate 3D7 is an example of a chloroquine-sensitive parasite isolate.

As used herein, “antimalarial-resistant isolate” or “antimalarial-resistant” refers the ability of a parasite to survive and/or multiply despite the administration and absorption of a drug given in doses equal to or higher than those usually recommended but within tolerance of the subject, for the duration of the time necessary for its normal action. An isolate that is resistant to an antimalarial is not susceptible to the biological effects of that antimalarial. For example, a chloroquine-resistant isolate is viable in the presence of chloroquine in amounts that would kill a drug sensitive isolate. P. falciparum isolates 7G8 and K1 are examples of chloroquine-resistant parasite isolates.

As used herein, “treat”, “treating” or “treatment” includes both therapeutic and prophylactic treatment. Therapeutic treatment includes reducing the symptoms associated with a disease or condition and/or increasing the longevity of a subject with the disease or condition. As used herein, “prevent”, “preventing” or “prevention” includes prophylactic treatment which delays the onset of a disease or condition in a subject at risk of developing the disease or condition or reducing the likelihood that a subject will then develop the disease or condition in a subject that is at risk for developing the disease or condition.

“Alkyl” used alone or as part of a larger moiety such as “haloalkyl” or “alkylamine” refers to a straight or branched, saturated aliphatic group having the specified number of carbons, typically having 1 to 12 carbon atoms. More particularly, the aliphatic group may have 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

“Alkylene” refers to a bivalent saturated straight-chained hydrocarbon, e.g., C₁-C₆ alkylene includes —(CH₂)₆—, —CH₂—CH—(CH₂)₃CH₃, and the like. “Bivalent means that the alkylene group is attached to the remainder of the molecule through two different carbon atoms.

“Alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

“Amino” means —NH₂; “alkylamine” and “dialkylamine” mean —NHR and —NR₂, respectively, wherein R is an alkyl group.

“Haloalkyl” includes mono, poly, and perhaloalkyl groups where the halogens are independently selected from fluorine, chlorine, bromine and iodine.

“Cycloalkyl” used alone or as part of a larger moiety such as “cycloalkylalkyl” refers to a monocyclic or polycyclic, non-aromatic ring system of 3 to 20 carbon atoms, 3 to 12 carbon atoms, or 3 to 9 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—C≡CH), propargyl (—CH₂C≡CH), pentynyl, hexynyl, and the like.

The compounds according to the present invention may be in free form, in the form of pharmaceutically acceptable salts, or in the form of physiologically acceptable, non-toxic salts. These salts may be obtained by reacting the respective compounds with physiologically acceptable acids and bases.

Examples of such salts include but are not limited to hydrochloride, hydrobromide, hydroiodide, hydrofluoride, nitrate, sulfate, bisulfate, pyrosulfate, sulfite, bisulfite, phosphate, acid phosphate, monohydrogenphosphate, dihydrogenphosphate, metaphosphate, pyrophosphate, isonicotinate, acetate, trifluoroacetate, propionate, caprylate, isobutyrate, lactate, salicylate, citrate, tartrate, oxalate, malonate, suberate, sebacate, mandelate, chlorobenzoate, methylbenzoate, dinitrobenzoate, phthalate, phenylacetate, malate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminium, calcium, lithium, magnesium, potassium, sodium, zinc, and diethanolamine, N,N′-dibenzylethylenediamine, chloroprocaine, choline, dicyclohexylamine, ethylenediamine, N-methylglucamine, and procaine salts.

### EXEMPLIFICATION

### Example 1

### Synthesis of Fluorophore-tagged antimalarials

Coumarin-labeled chloroquine (CM-CQ) and Compounds 8, 13 and 16

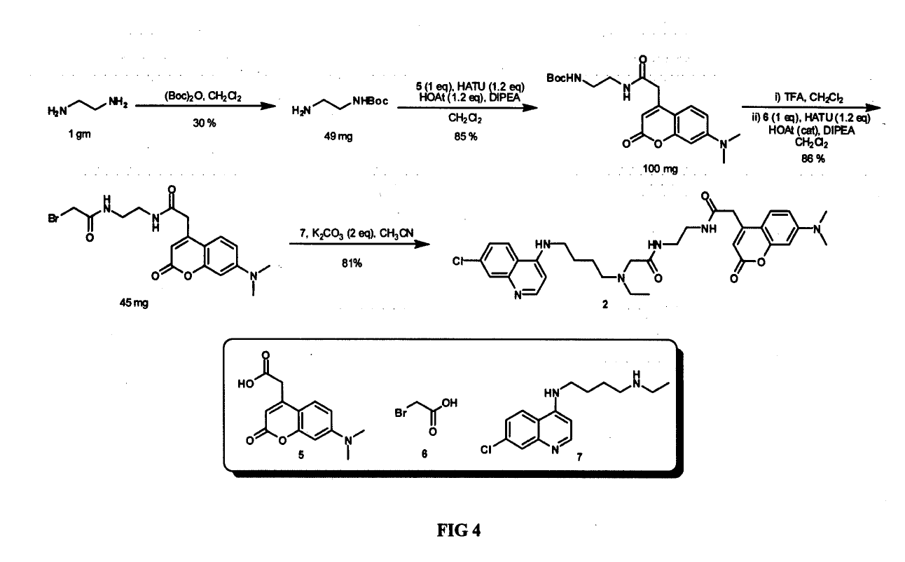

A fluorescent-labeled CQ analog based on the blue fluorescence exhibited by a biologically-benign coumarin (CM) was designed and synthesized (See FIG. 4).

Under a standard synthetic procedure (See Prusov, E., and Maier, M. E. (2007) Tetrahedron 63, 10486-10496), using flame-dried glass apparatus under nitrogen atmosphere, des(N-ethyl)-chloroquine (25.0 mg, 0.09 mmol), i.e., N-(7-chloro-4-quinolinyl)-N′-ethyl-1,4-diaminobutane (See Natarajan, J. K., et al., (2008) J Med Chem 51, 3466-3479), was dissolved in anhydrous acetonitrile (5.0 ml). Finely powdered and pre-dried potassium carbonate (24.8 mg, 0.2 mmol) was then added, followed by the addition of an anhydrous acetonitrile solution (1.0 ml) of the coumarin α-bromoester (44.2 mg, 0.1 mmol), i.e., 2-bromo-N-(2-(2-(7-(dimethylamino)-2-oxo-2H-chromen-4-yOacetamido)ethyl)-acetamide (See Alexander, M. D., et al., (2006) Chembiochem 7, 409-416). The mixture was stirred for 13 hours at room temperature, then filtered and the filtrate evaporated to give a yellow residue. The residue was then purified by flash column chromatography (230-400 mesh silica gel) to give CM-CQ (45.0 mg, 0.074 mmol) in 82% yield.

NMR measurements of CM-CQ were recorded on a 300 MHz (for ¹H) and 500 MHz (for ¹³C) Broker spectrometer fitted with a pulse-field gradient probe. The residual resonance of deuterated solvents was used as an internal reference. ¹H NMR (300 MHz, MeOD) δ: 0.87 (t, J=6.4 Hz, 3H), 1.52-1.57 (m, 2H), 1.62-1.74 (m, 2H), 2.41 (t, J=7.0 Hz, 2H), 2.49 (q, J=7.0 Hz, 2H), 2.97 (bs, 8H), 3.21 (m, 2H), 3.38-3.40 (m, 4H), 3.63 (s, 2H), 5.98 (s, 1H), 6.34 (d, J=23 Hz, 1H), 6.43 (d, J=5.8 Hz, 1H), 6.55 (dd, J=2.6 Hz, 9.1 Hz, 1H), 7.25 (dd, J=2.0 Hz, 8.8 Hz, 1H), 7.42 (d, J=8.8 Hz, 1H), 7.71 (d, J=2.0 Hz, 1H), 7.99 (d, 9.1 Hz, 1H), 8.29 (d, 5.8 Hz, 1H). ¹³C NMR (125 MHz, MeOD): δ 12.1, 26.2, 27.1, 40.0, 40.1, 40.2, 44.0, 47.4, 50.0, 55.7, 58.7, 98.6, 99.6, 109.7, 110.3, 110.5, 118.7, 124.5, 125.8, 126.8, 127.3, 136.2, 149.4, 152.2, 152.7, 154.6, 157.1, 164.2, 171.5, 175.2.

Analytical HPLC and mass spectrometry of CM-CQ was carried out on a Shimadzu LC-IT-TOF system equipped with an autosampler, using reverse-phase Phenomenex Luna 5 μm C18, 50×3.0 mm column. Solvent mixtures of 0.1% TFA/H₂O (solvent-A) and 0.1% TFA/CH₃CN (solvent-B) were used as co-eluents under a linear gradient of 1% to 100% of solvent-B (over 45 min; flow rate of 0.5 ml/min). This gave a purity of CM-CQ of 98% with a found m/z [M+1] of 607.277 (calculated for C₃₂H₃₉ClN₆O₄: 607.280).

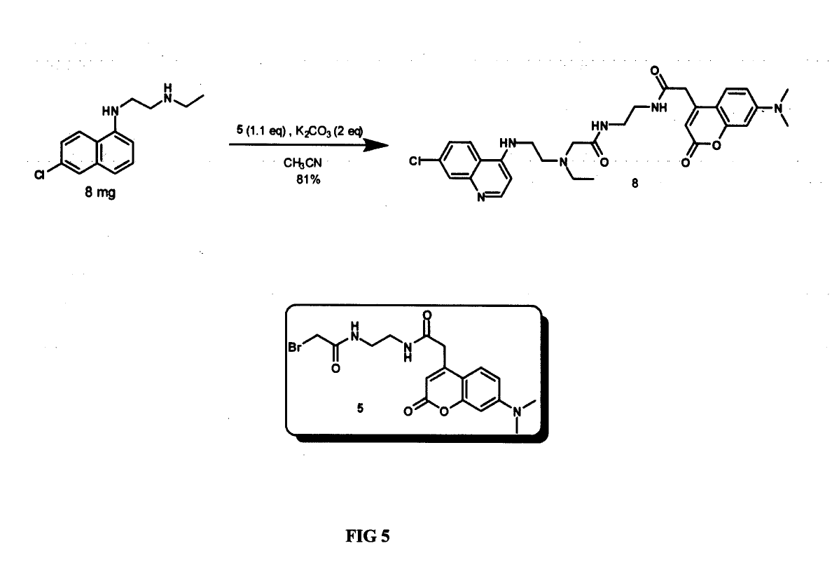

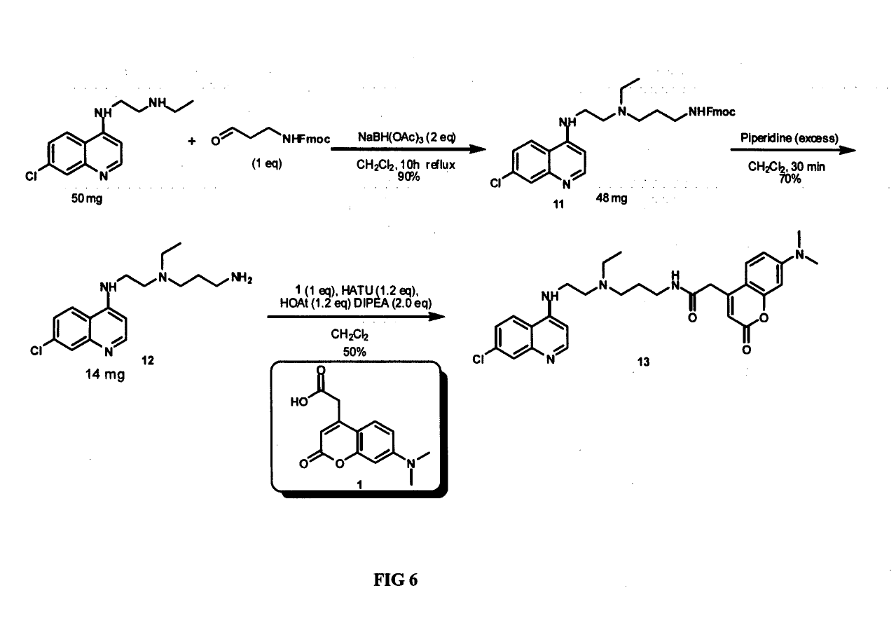

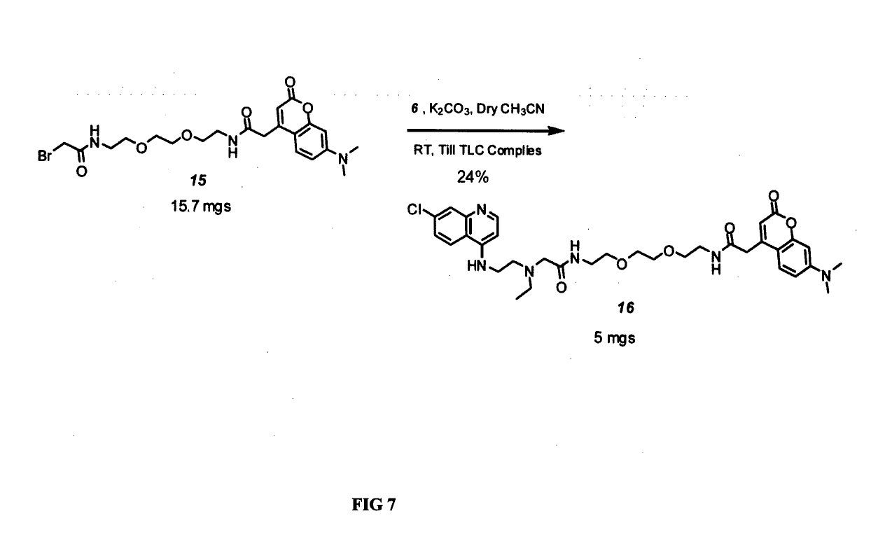

Additional fluorophore-tagged antimalarials were synthesized as depicted in FIG. 5 (Compound 8), FIG. 6 (Compound 13, the reductive amination step was adapted from Ekoue-Kovi et al. Bioorg. Med. Chem. 2009, 17, 270-283, the teachings of which are incorporated herein by reference) and FIG. 7 (Compound 16).

### Example 2

### Use of CM-CQ in Cell Localization Studies

Confocal bioimaging assays were carried out with P. falciparum-infected red blood cells (RBCs) exposed to increasing concentration of CM-CQ. The drug accumulates in different cellular compartments depending on the concentration of CM-CQ as seen in FIG. 1.

The cellular distribution of CM-CQ in 7G8 (CQ-resistant) and K1 (CQ-resistant) will be investigated by a similar protocol. The localization pattern should differ between drug resistant and sensitive malaria isolates. Therefore, the localization of the fluorophore-tagged antimalarial could be used to distinguish between resistant and sensitive isolates.

### Example 3

### Use of CM-CQ in β-hematin Binding Assays

β-hematin binding assays will be carried out with CM-CQ and CQ. CQ and other quinolones are known to bind to β-hematin, and the extent of binding can be quantified spectrophotometrically (See Auparakkitanon et al. Antimicrobial Agents and Chemotherapy 2003 47:3708-3712). Exposure of β-hematin to CM-CQ is hypothesized to result in comparable binding coefficients when compared to CQ-hematin interaction.

### Example 4

### Use of CM-CQ in Apoptosis Study of P. falciparum

CM-CQ was used to investigated the inducement of programmed cell death (PCD) in parasites cultures by chloroquine.

### Materials and Methods

Plasmodium falciparum culture. A CQ-sensitive laboratory strain of P. falciparum, 3D7, was cultured continuously with minor modifications to the method previously described (See Trager, W., and Jensen, J. B. (2005) J. Parasitol 91, 484-486). Parasite cultures were maintained in 75 cm² or 125 cm² flasks using malaria culture media (MCM) consisting of RPMI 1640 supplemented with 0.5% (w/v) albumax I (Invitrogen), 0.005% (w/v) hypoxanthine, 0.03% (w/v) L-glutamate, 0.25% (w/v) gentamycin and 2.5% human erythrocytes. Flasks were gassed with 3% CO₂, 4% O₂ and 93% N₂ and incubated at 37° C. in a dark incubator. Culture medium was replenished during subculturing on alternate days. Thin Giemsa smears were made to determine parasitemia before subculture and at the start of each experiment. Sorbitol synchronization (See Lambros, C., and Vanderberg, J. P. (1979) J Parasitol 65, 418-420) was carried out weekly to select for ring-stage parasites. Saponin enrichment (See Beaumelle, B. D., et al., (1987) J. Parasitol 73, 743-748) was carried out prior to staining with CaspaTag to liberate the parasites from host erythrocytes. Cultures of approximately 10% parasitemia were used, with parasites being in the late-ring stage (22-26 hours post-invasion) during the administration of inhibitor and drugs unless otherwise stated.

Drug preparation and treatments. Chloroquine diphosphate (CQ, Sigma-Aldrich) was dissolved in PBS and filter-sterilized to obtain working solutions of 300 μM, and stored in the dark at 4° C. A new batch of CQ working solution was made before each experiment. Parasitized erythrocytes were incubated for 8 or 10 hours in 30 μM of CQ and washed twice with MCM prior to assaying for apoptotic features.

Mitochondria trans-membrane potential assay. Cell permeable lipophilic cation probe JC-1 (Molecular Probes), also known as 5,5′, 6,6′-tetrachloro-1,1′,3,3′-tetraethyl-benzimidazolylcarbocyanine iodine, normally has green fluorescence (525 nm) but aggregates at high transmembrane potential of functional mitochondria to emit an orange-red fluorescence (excitation maximum at 590 nm) (See Smiley, S. T., et al., (1991) Proc Natl Acad Sci USA 88, 3671-3675). A final concentration of 6 μM of JC-1 was added to 0.5 ml of treated parasitized erythrocytes and incubated at 37° C. in a dark humidified candle jar for 30 minutes. Cells were washed twice with PBS and resuspended to the original volume before being analyzed by flow cytometry. PE:FITC ratios were obtained to determine the extent of mitochondrial dysregulation.

Inhibitory concentration (IC₅₀) determination. Parasitized cultures (ring stage) diluted with fresh erythrocytes and MCM to 2% parasitemia and 1.25% hematocrit were incubated with various concentrations of commercial CQ (Sigma), coumarin (CM, i.e., 7-dimethyl amino-4-coumarinacetic acid) and CM-CQ for 48 hours. To determine parasitemia, cultures were stained with Hoechst 33342, as described above, and analyzed by flow cytometry. The IC₅₀ will be determine by a similar protocol for CQ-resistant isolates, such as K1 and 7G8.

Flow Cytometric Analyses. Cell numbers and fluorescence intensity after staining with JC-1 were assayed by a flow cytometer (DAKO Cytomation Cyan ADP), equipped with an argon-ion laser tuned to 488 nm and a violet laser tuned to 405 nm. Green and red fluorescence was detected using 525 and 595 nm band-pass filters respectively. The flow cytometer was adjusted for forward and side scattering to accommodate infected erythrocytes. Between 10,000 to 20,000 infected erythrocytes or enriched parasites were analyzed from each sample and its respective duplicate to ensure reliability. At least three independent experiments were performed unless otherwise stated.

Confocal microscopy. Confocal imaging was done using Olympus® Fluoview FV1000™ (Japan) equipped with solid state and Argon ion lasers tuned to 405 nm and 488 nm respectively. Images were captured using Olympus® Fluoview version 1.6b.

Statistical analyses. All data shown are means ±SEM. Statistical difference between percentages of stained cells was measured using one-sided paired t-test for means. Significantly different results (p<0.05) were highlighted.

### Results

Mitochondrial membrane potential assay. The accumulation of JC-1 J-aggregates in uninduced cells showed that about 90% of the parasites contained functional mitochondrion with transmembrane potential (ΔΨₘ). Fluorescence microscopy confirmed the specificity of the aggregated mitochondrial stain. Treatment with CQ or CM-CQ caused a dose-dependent decline in this proportion to approximately 50% and 60% respectively.

Inhibitory Concentration. The IC₅₀ (50% inhibitory concentration) of CM-CQ for P. falciparum strains 3D7 (CQ-susceptible) indicated that the biological activity of CQ is maintained even with attachment to coumarin. To ascertain the biological efficacy of CM-CQ, IC₅₀ curves were measured and the inhibitory efficiencies of the chloroquine compounds determined (FIG. 2). The IC₅₀ values of commercial CQ and CM-CQ were determined to be 42.6873 nM and 349.324 nM respectively. Coumarin (CM) had no detectable inhibitory effect at the tested concentrations and was not cytotoxic.

The biological activity of the CM-CQ in 7G8 (CQ-resistant) and K1 (CQ-resistant) will be investigated by a similar protocol. Because 7G8 and K1 isolates are resistant to CQ, it is hypothesized that the IC₅₀ of CM-CQ will be substantially higher for these two isolates than for 3D7. Resistance to CQ in K1 strain is reportedly reversible by verapamil, whereas that for 7G8 is not affected by this compound (See Chen et al. Antimicrobial Agents and Chemotherapy 2003 47:3500-3505). As such, pre-treatment of parasitized cultures with verapamil is hypothesized to decrease the IC₅₀ value of K1 while that of 3D7 and 7G8 should remain relatively unchanged even in the presence of verapamil, suggesting that resistance of K1 and 7G8 to CQ also protects these strains from CM-CQ.

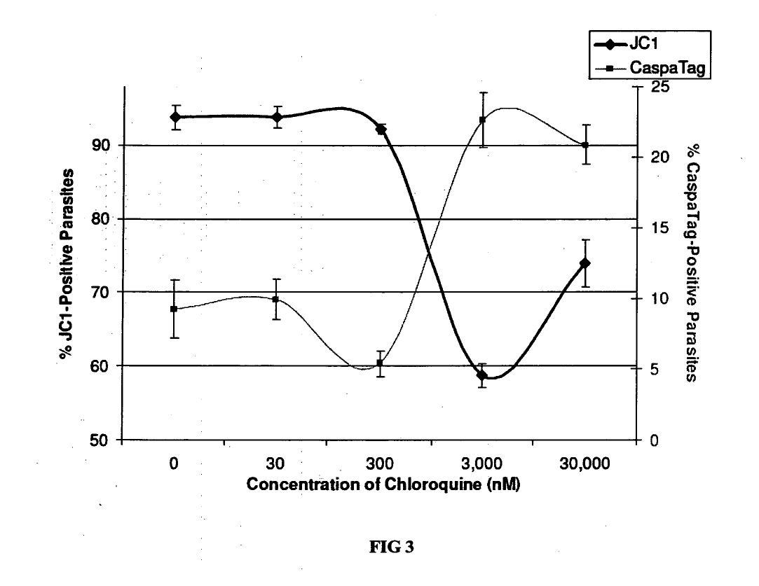

Dose-dependent effect of chloroquine. JC-1 and CaspaTag assays were used to investigate programmed cell death (PCD) features at different concentrations of CQ. At low nanomolar concentrations, there were basal levels of PCD features but at micromolar concentrations of CQ, there was a large increase in the proportion of cells displaying mitochondria outer membrane permeabilization (MOMP) and activation of caspase-like proteases (FIG. 3). Confocal microscopy was then employed to assay for concentration-dependent differences in the cellular localization of CM-CQ. It was observed that CM-CQ accumulated in the hemazoin-containing food vacuole at nanomolar concentrations, which is in agreement with conventionally accepted mechanism of CQ's antimalarial activity (See Foley, M., and Tilley, L (1998) Pharmacol Ther 79, 55-87). At micromolar concentrations however, the labeled drug accumulated non-specifically throughout the parasite's cytoplasm. Confocal microscopy showed by JC-1 staining that the function of the mitochondrion of a parasite is reduced upon exposure to coumarin-labeled chloroquine (CM-CQ). No accumulation of CM-CQ is detected at 30 nM, but at 300 nM, CM-CQ localizes in the hemazoin-containing food vacuole of the parasite. Accumulation of CM-CQ within the parasite is non-specific at concentrations exceeding 3 μM and localizes throughout the parasite's cytoplasm. At these concentrations, CM-CQ also induced mitochondria-outer membrane permeabilization resulting in mitochondria that were not clearly defined.

### Discussion

Localization studies using CM-CQ, a fluorescent-labeled analog of chloroquine, showed a shift in the localization of the drug from the food vacuole at nanomolar concentrations, to the cytoplasm of the parasite at micromolar concentrations. This suggests that the PCD features observed are brought about by CQ accumulation and by the subsequent activation of PCD mediators in the cytoplasm.

### Example 5

### Use of CM-CQ in Studies with Chemo-sensitizers

### Methods

Synchronized malaria parasite cultures (mid-ring stage of Plasmodium falciparum) of approximately 10% parasitemia and 2.5% hematocrit were pre-treated with either a vehicle control (PBS) or 10 uM of known chemo-sensitizers verapamil (VPM), propranolol (PPL), diltiazem (DTZ), chlorpromazine (CPZ), desipramine (DSP), promethazine (PMZ) or chlorpheneramine (CPR) for 30 min at 37° C. Subsequently, 6 uM of Coumarin-Chloroquine (CM-CQ) was added to the samples for 10 hrs and left to incubate in the dark at 37° C. To ascertain the exact parasitemia of each condition, cultures in duplicate wells were stained with Hoechst just before flow cytometric analysis and fluorescence plate measurements. The proportion of CM-CQ-stained parasites was the ratio of the proportion of CM-CQ-stained infected erythrocyte culture over the proportion of Hoechst-positive erythrocytes.

### Molecular Structure of CM-CQ:

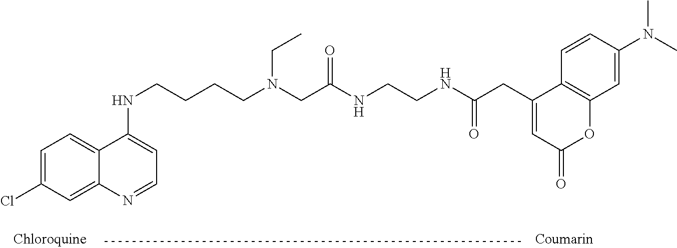

### Results of Flow Cytometry Analysis

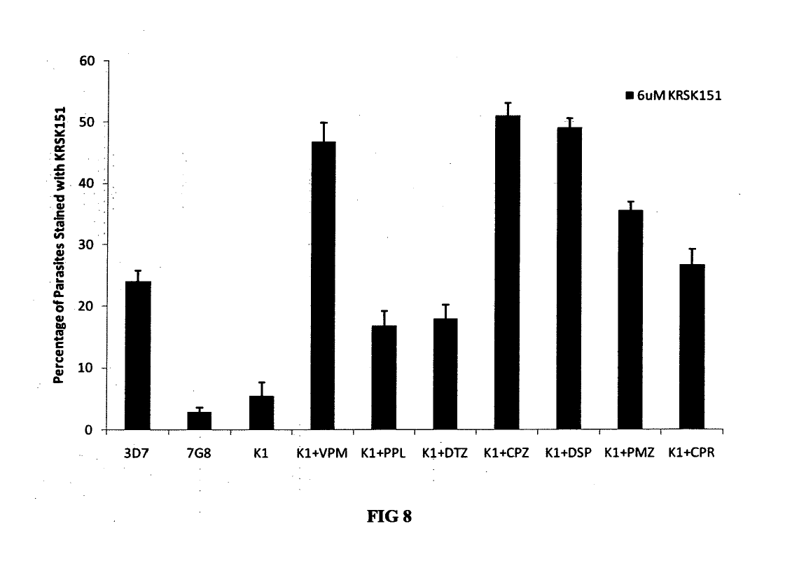

Flow cytometry analysis revealed that chloroquine (CQ)-susceptible strain 3D7 showed different levels of CM-CQ accumulations compared to CQ-resistant strains 7G8 and K1 (p<0.05 for both). Pre-treatment of K1 with VPM, CPZ, DSP and PMZ significantly increased CM-CQ accumulation (p<0.001 for all). This increase was also detected when K1 was pre-treated with CPR (p<0.05) but not when cells were pre-treated with PPL or DTZ. (Data represented means from two separate experiments.) FIG. 8 shows percentages of parasites stained with CM-CQ for chloroquine (CQ)-susceptible strain 3D7, CQ-resistant strains 7G8 and K1 and K1 strain sensitized with seven known chemo-sensitizers described above, measured by flow cytometry.

### Results of Fluorescent Plate Reader Analysis

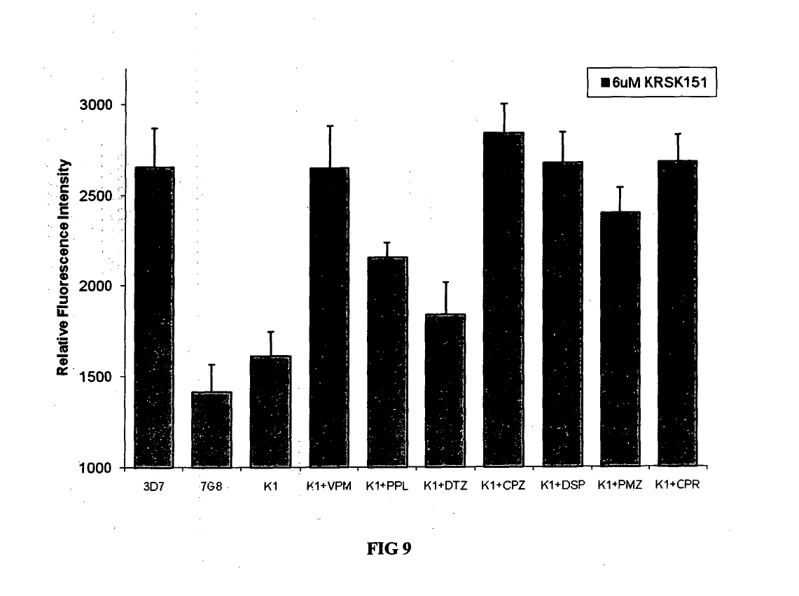

Analysis by Fluorescent plate reader revealed that chloroquine (CQ)-susceptible strain 3D7 showed different levels of CM-CQ accumulations compared to CQ-resistant strains 7G8 and K1 (p<0.001 for both). Pre-treatment of K1 with VPM, CPZ, DSP and CPR significantly increased CM-CQ accumulation (p<0.001 for all). This increase was also detected when K1 was pre-treated with PMZ (p<0.01) but not when cells were pre-treated with PPL or DTZ. (Data represented means from two separate experiments.) FIG. 9 shows relative fluorescent intensity for (CQ)-susceptible strain 3D7, CQ-resistant strains 7G8 and K1 and K1 strain sensitized with seven known chemo-sensitizers described above, measured by direct plate fluorescent readout.

### CONCLUSIONS

CM-CQ was useful in distinguishing between CQ-sensitive and CQ-resistant strains of P. falciparum. Further, CM-CQ was able to reliably detect 5 out of the 7 chemo-sensitizers tested through increased fluorescence accumulation in infected cultures using either flow cytometry (FIG. 8) or fluorescence plate measurements (FIG. 9).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A compound for use in malarial research, comprising a coumarin-tagged antimalarial, wherein the coumarin is optionally substituted and is connected to the antimalarial through a linker.

2. A compound comprising a coumarin-tagged antimalarial, comprising coumarin connected to the antimalarial through a linker, wherein the coumarin and linker are represented by the following structural formula: 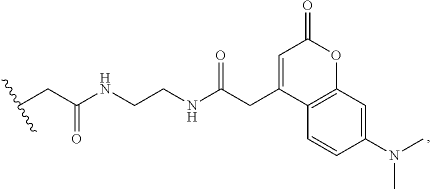
or salts thereof.

3. The compound of claim 1, wherein the antimalarial molecule is chloroquine, a chloroquine derivative or a salt thereof.

4. A compound represented by the following structural formula: 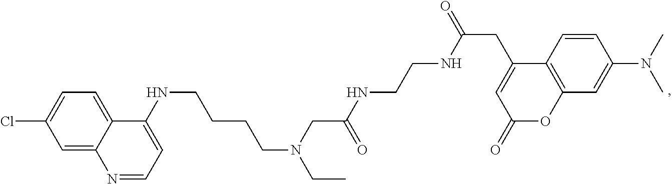
or a salt thereof.

5. A compound represented by the following structural formula: 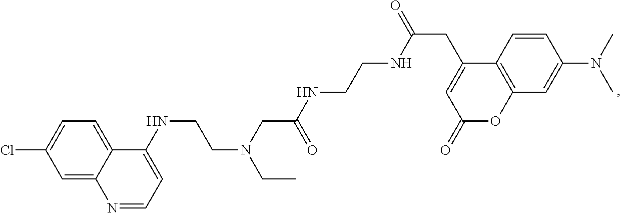
or a salt thereof.

6. A compound represented by the following structural formula: 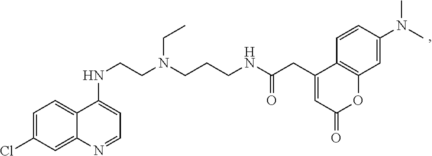
or a salt thereof.

7. A compound represented by the following structural formula: 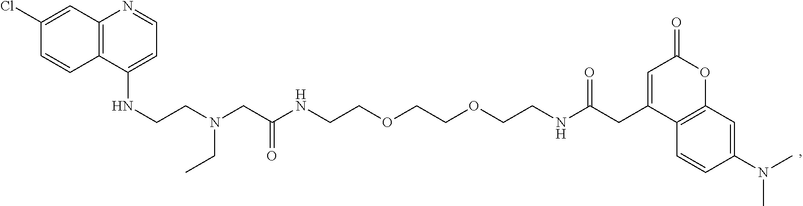
or a salt thereof.

8. A method for identification of a target in a cell, comprising the steps of:
exposing the cell to the fluorophore-tagged antimalarial of claim 1;
allowing the fluorophore-tagged antimalarial bind to its target;
capturing the fluorophore-tagged antimalarial bound to the target on an immobilized affinity ligand specific for the fluorophore tag; and
identifying the target bound to the fluorophore-tagged antimalarial.

9. A method for identification of a target in a cell, comprising the steps of:
exposing the cell to the fluorophore-tagged antimalarial of the following structural formula: 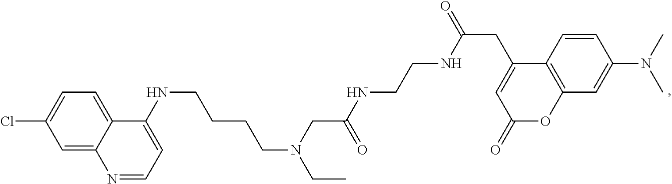
or a salt thereof;
allowing the fluorophore-tagged antimalarial bind to its target;
capturing the fluorophore-tagged antimalarial bound to the target on an immobilized affinity ligand specific for the fluorophore tag; and
identifying the target bound to the fluorophore-tagged antimalarial.

10. The method of claim 9, wherein the target is identified by N-terminal or internal protein sequencing, mass spectrometry, gel electrophoresis, or gene silencing with siRNA or RNAi.

11. A method for determining the location of a fluorophore-tagged antimalarial within or on a cell, comprising the steps of:
exposing the cell to the fluorophore-tagged antimalarial of claim 1; and
assaying the cell to determine the location of the fluorophore-tagged antimalarial within or on the cell.

12. A method for determining the location of a fluorophore-tagged antimalarial within or on a cell, comprising the steps of:
exposing the cell to the fluorophore-tagged antimalarial of the following structural formula: 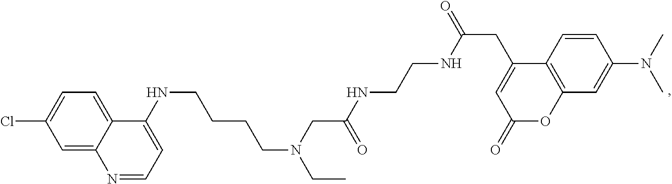
or a salt thereof; and
assaying the cell to determine the location of the fluorophore-tagged antimalarial within or on the cell.

13. The method of claim 12, wherein the cell is visualized by confocal microscopy.

14. The method of claim 12, wherein the cell is visualized by fluorescence microscopy.

15. The method of claim 12, wherein the cell is assayed by flow cytometry.

16. A method of preparing a fluorophore-tagged antimalarial represented by the following structural formula: 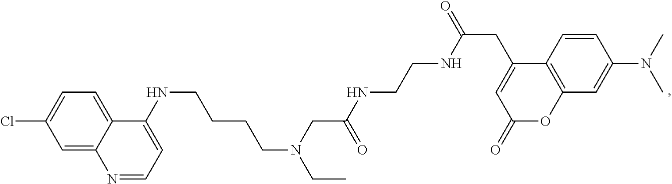
or a salt thereof, said method comprising the step of:
reacting a compound represented by the following structural formula: 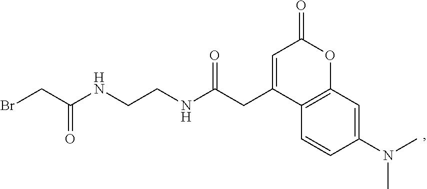
or a salt thereof with an antimalarial represented by the following structural formula: 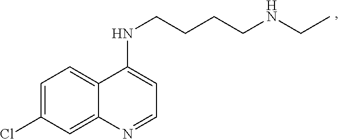
or a salt thereof, thereby forming the fluorophore-tagged antimalarial.

17. A method for identification of a compound that can alter resistance to chloroquine in a resistant strain of Plasmodium, comprising:
a) exposing a chloroquine resistant Plasmodium parasite to a test compound;
b) adding the fluorophore-tagged antimalarial of the following structural formula: 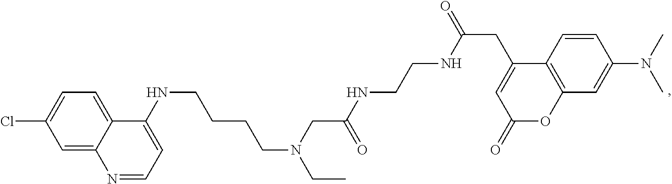
or a salt thereof to the exposed parasite of step a); and
c) measuring fluorescence signal, wherein an increase in the fluorescence signal from a baseline measurement is indicative of a compound that can alter chloroquine resistance.

18. A method of identification of a chloroquine resistant strain of Plasmodium, comprising:
a) exposing a chloroquine resistant strain of Plasmodium to the fluorophore-tagged antimalarial of the following structural formula: 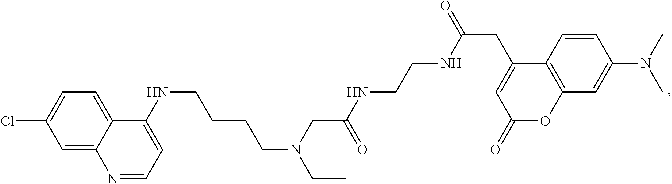
or a salt thereof; and
b) measuring fluorescence signal, wherein a decrease in the fluorescence signal from a baseline measurement is indicative of the chloroquine resistant strain.

19. The method of claim 17, wherein the parasite is visualized by confocal microscopy.

20. The method of claim 17, wherein the parasite is visualized by fluorescence microscopy.

21. The method of claim 17, wherein step (b) is measured by flow cytometry.
